(19) United States
(12) Patent Application Publication
(10) Pub. No.: US 2018/0014783 A1
(43) Pub. Date: Jan. 18, 2018

(54) Wearable patch having reliable conductive contacts for measuring electrical signals

(71) Applicant: VivaLnk, Inc., Santa Clara, CA (US)

(72) Inventors: Wei Shi, San Jose, CA (US); Jiang Li, Cupertino, CA (US)

(21) Appl. No.: 15/690,731

(22) Filed: Aug. 30, 2017

Related U.S. Application Data
(63) Continuation-in-part of application No. 15/209,392, filed on Jul. 13, 2016.

(51) Int. Cl. A61B 5/00; A61B 5/0408

(52) U.S. Cl. CPC A61B 5/6833; A61B 5/04087; A61B 5/0024; A61B 5/04085; A61B 5/0004; A61B 2560/0412; A61B 2562/0209; A61B 2562/164

(57) ABSTRACT

A wearable patch for measuring electrical signals from a user's body includes a main substrate, a circuit substrate on the main substrate, the circuit substrate comprising a conductive circuit; a first conductive electrode circuit in electric connection with the conductive circuit and including a portion on a lower surface of the main substrate; and an adhesive-conductive assembly attached to the lower surface of the main substrate. The adhesive-conductive assembly includes a support layer having a first opening and a conductive gel layer inserted into the first opening and configured to be in contact with a user's skin, a first adhesive layer to bond to portions of upper surfaces of the conductive gel layer and a portion of an upper surface of the support layer, and a second adhesive layer to bond to portions of lower surfaces of the conductive gel layer and a portion of a lower surface of the support layer.

### BACKGROUND OF THE INVENTION

The present application relates to wearable electronic devices, and in particular, to wearable patches for measuring electric signals from human body.

Electronic patches can be used for tracking objects and for performing functions such as producing sound, light or vibrations, and so on. As applications and human needs become more sophisticated and complex, electronic patches are required to perform a rapidly increasing number of tasks. Electronic patches are often required to be conformal to curved surfaces, which in the case of human body, can vary overtime.

Electronic patches can communicate with smart phones and other devices using Wi-Fi, Bluetooth, Near Field Communication (NFC), and other wireless technologies. NFC is a wireless communication standard that enables two devices to quickly establish communication within a short range around radio frequency of 13.56 MHz. NFC is more secure than other wireless technologies such as Bluetooth and Wi-Fi because NFC requires two devices in close proximity (e.g. less than 10 cm). NFC can also lower cost comparing to other wireless technologies by allowing one of the two devices to be passive.

Bluetooth is another wireless communication standard for exchanging data over relatively longer distances (in tens of meters). It employs short wavelength UHF radio waves from 2.4 to 2.485 GHz from fixed or mobile devices. Bluetooth devices have evolved to meet the increasing demand for low-power solutions that is required for wearable electronics. Benefited from relatively longer reading distance and active communication, Bluetooth technologies allow wearable patches to continuously monitoring vital information without human interference, which is an advantage over NFC in many applications.

Wearable patch (or tag) is an electronic patch that can be worn by a person. A wearable patch is required to stay on user's skin and operate for an extended period from hours to months. A wearable patch can contain a micro-electronic system that can be accessed using NFC, Bluetooth, Wi-Fi, or other wireless technologies. A wearable patch can include different sensors for measurements such as vital signs monitoring, motion track, skin temperature measurements, and ECG detection.

Despite recent development efforts, conventional wearable devices still face several drawbacks: their rigid substrates are very difficult to conform to curved surfaces and may not provide adequate comfort for users. They may not stay attached to user's body for the required length of time. Moreover, conventional wearable devices are often not robust enough to sustain repeated elongations during the movements of the user's body. Under stress, different layers in wearable patches can break or delaminate rendering the patches inoperable. Additionally, the rigid polymer substrates in conventional wearable patches do not allow much breathability to the skin. The build-up of sweat and moisture can cause discomfort and irritation to the skin, especially after wearing it for an extended period.

There is therefore a need for a durable device that can reliably measure human organs' electrical signals and are comfortable for users to wear.

### SUMMARY OF THE INVENTION

The presently disclosure attempts to address the aforementioned limitations in conventional wearable patches. The disclosed wearable patches are highly compliant, stretchable, and flexible, which makes them comfortable to wear, while also being able to support circuits, chips, and other electronic components. The disclosed wearable patches can change physical shapes and dimensions to relieve stresses such as repeated elongations, therefore increasing durability of the wearable patches as well as provide comfort to the users. The disclosed wearable patches can stay attached to skin for a long period while enduring muscle movements.

Furthermore, the disclosed wearable patches include flexible and stretchable electric contacts for measuring human organ electrical activities such as ECG, EEG, EMG, etc. The disclosed wearable patches can reliably measuring electrical signals in human body. Additionally, the disclosed wearable patches can eliminate the requirement for applying a conductive gel on the user skin for some conventional wearable patches.

Importantly, the disclosed wearable patches include an adhesive conductive assembly that can be made of low cost materials. The adhesive conductive assembly is replaceable, which allows the more expensive portions of the wearable patch to be reused for longer time.

In one general aspect, the present invention relates to a wearable patch for measuring electrical signals from a user's body that includes a main substrate; a circuit substrate on the main substrate, the circuit substrate comprising a conductive circuit; a semiconductor chip in electric connection with the conductive circuit; a first conductive electrode circuit in electric connection with the conductive circuit, wherein the first conductive electrode circuit includes a portion on a lower surface of the main substrate; and an adhesive-conductive assembly attached to the lower surface of the main substrate, which includes: a support layer comprising a first opening; a first conductive gel layer inserted into the first opening and configured to be in contact with a user's skin; a first adhesive layer that can bond to portions of upper surfaces of the first conductive gel layer and a portion of an upper surface of the support layer, wherein the first adhesive layer comprises an upper surface that can bond to the lower surface of the main substrate, which enables an electric contact to be formed between the first conductive electrode circuit and the first conductive gel layer; and a second adhesive layer that can bond to portions of lower surfaces of the first conductive gel layer and a portion of a lower surface of the support layer, wherein the second adhesive layer comprises a lower surface that can be attached to a user's skin, which allows the first conductive electrode circuit to pick up electric signals from the user's body via the first conductive gel layer.

Implementations of the system may include one or more of the following. The wearable patch can further include: a second conductive electrode circuit in electric connection with the conductive circuit, wherein the second conductive electrode circuit includes a portion on a lower surface of the main substrate, wherein the support layer can include a second hole opening, wherein the adhesive-conductive assembly can further include: a second conductive gel layer inserted into the second opening and that can be in contact with a user's skin, wherein the first adhesive layer can further bond to portions of upper surfaces of the second conductive gel layer, wherein the upper surface on the first adhesive layer can bond to the lower surface of the main substrate, which enables an electric contact to be formed between the second conductive electrode circuit and the second conductive gel layer; and a second adhesive layer that can further bond to portions of lower surfaces of the second conductive gel layer, wherein the second adhesive layer includes a lower surface that can be attached to a user's skin, which allows the second conductive electrode circuit to pick up electric signals from the user's body via the second conductive gel layer. The second conductive gel layer can protrude above and below the support layer, wherein the second conductive gel layer includes a lower surface that can be in contact with a user's skin. The main substrate can include a via through which the second conductive electrode circuit forms a connection between portions on the lower surface and an upper surface of the main substrate. The first conductive gel layer can protrude above and below the support layer, wherein the first conductive gel layer includes a lower surface that can be in contact with a user's skin. The main substrate can include a via through which the first conductive electrode circuit forms a connection between portions on the lower surface and an upper surface of the main substrate. The first conductive gel layer can include hydro gel. The upper surface and the lower surface of the second adhesive layer can include an adhesive. The upper surface and the lower surface of the first adhesive layer can include an adhesive. The first adhesive layer can be wider than the support layer to allow the lower surface of the first adhesive layer to attach to a user's skin. The semiconductor chip can wireless communicate with an external device in response to the electric signals picked up by the first conductive electrode circuit from the user's body. The wearable patch can further include a cover formed on the main substrate, portions of the first conductive electrode circuit, and the circuit substrate.

These and other aspects, their implementations and other features are described in detail in the drawings, the description and the claims.

### BRIEF DESCRIPTION OF THE DRAWINGS

FIG. 1 illustrates the usage of wearable patches attached to a user's skin.

FIG. 2 is a cross-sectional view of a compliant wearable patch having reliable conductive contacts to a user's skin in accordance with some embodiments of the present invention.

FIG. 3 is a cross-sectional view of another compliant wearable patch comprising replaceable electrode contacts in accordance with some embodiments of the present invention.

FIG. 4 is a bottom planar view of the compliant wearable patch in FIG. 3.

FIG. 5 is a cross-sectional view of another compliant wearable patch comprising replaceable electrode contacts in accordance with some embodiments of the present invention.

FIG. 6 is a bottom planar view of the compliant wearable patch in FIG. 5.

### DETAILED DESCRIPTION OF THE INVENTION

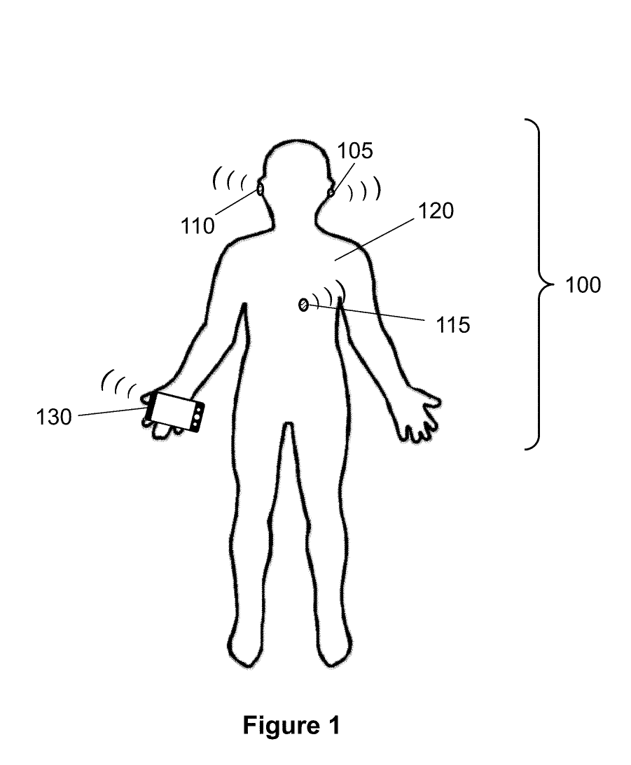

Referring to FIG. 1, a system 100 of wearable patches 105, 110, 115 are attached to the body or the skin of a user 120 for measuring body vital signs and other signals. The wearable patches 105, 110, 115 can be placed on the ears, the forehead, the chest, the hands, the shoulder, the waist, the leg, or the foot, under the armpit, around the wrist, on or around the arm, or on other parts of the user's body. The wearable patches 105, 110, 115, as described in the examples below, include sensors that can sense a variety of signals such as temperature, electric voltage, blood pressure, heart pulse, force, acceleration, blood oxygen level, blood glucose level, etc. In the present disclosure, the term “wearable patch” can also be referred to as “wearable sticker”, “wearable tag”, or “wearable band”, etc.

The wearable patches 105, 110, 115 can operate individually, or in a group to provide certain desired treatment or measurement. For example, one of the wearable patch 105, 110, 115 can wrap around a user's ear for applying an electric field through certain location of the ear. Moreover, the wearable patches 105, 110, 115 can be attached to different parts of a user's body such as on the two ears or the two temples of the user 120, which allows a low electric voltage signal to be measured or applied across the user's head or other parts of the user's body.

The system 100 also includes a wireless control device 130 that can wirelessly exchange data with the wearable patches 105, 110, 115. The wireless communications can be conducted using Wi-Fi, Bluetooth, Near Field Communication (NFC), and other wireless technologies. The wireless control device 130 can be a portable mobile device, which can be carried by the user 120. The wireless control device 130 can also be a stationary device that can be placed at home or office where the user 120 may stay for an extended period. The portable mobile device can be implemented with specialized hardware and software units built in a smart phone, a tablet computer (including devices such as iPod), or a dedicated health or sport monitoring device. The wireless control device 130 can be in communication with a network server in which a user account is stored for the user.

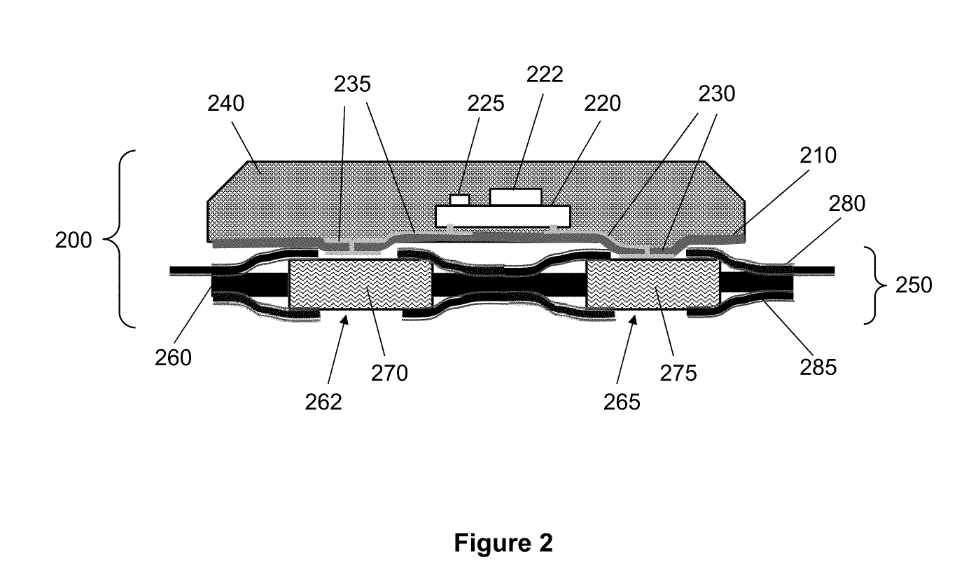

To overcome the above-described drawbacks in the conventional wearable patches, referring to FIG. 2, a compliant wearable patch 200 includes a main substrate 210 and a circuit substrate 220 on the main substrate 210. The compliant wearable patch 200 is suitable to be used as the wearable patches 105, 110, 115 in the system 100 (FIG. 1). A semiconductor chip 222, an antenna 225, and other electronic components such as a battery (not shown) are mounted on the circuit substrate 220 and in connection with a circuit (not shown) in the circuit substrate 220. A conductive electrode circuit 230 is formed on and under the main substrate 210 with its upper and lower portions connected by a conductive via through the main substrate 210. Similarly, a conductive electrode circuit 235 is formed on and under the main substrate 210 with its upper and lower portions connected by a conductive via through the main substrate 210. The conductive electrode circuits 230, 235 can be made of a conductive material such as Ag/AgCl, which can be formed respectively by coating the conductive material on and under the main substrate 210 and filling vias with the conductive material. The conductive electrode circuits 230, 235 are in connection with the circuit in the circuit substrate 220. A soft cover 240 is formed on the main substrate 210, portions of the conductive electrode circuits 230, 235, and the circuit substrate 220. The soft cover 240 can be formed by a foam or silicone.

The compliant wearable patch 200 also includes an adhesive-conductive assembly 250 that includes a soft support layer 260 having openings 262 and 265. The soft support layer 260 can be made of a soft or foam material such as polyurethane, polyethylene, fabric, etc. Conductive gel layers 270, 275 are respectively inserted into the openings 262, 265. The conductive gel layers 270, 275, which can be formed by a soft and sticky material such as hydro gel, are protruded above and below the support layer 260. A double-sided adhesive layer 280 is bonded to portions of the top surfaces of the conductive gel layers 270, 275 and the soft support layer 260. The upper surface of the double-sided adhesive layer 280 is attached to the lower surface of the main substrate 210. The double-sided adhesive layer 280 includes holes that expose portions of the upper surfaces of the conductive gel layers 270, 275, which respectively form electric contacts with the lower portions of the conductive electrode circuits 230, 235. Another double-sided adhesive layer 285 is bonded to portions of the lower surfaces of the conductive gel layers 270, 275 and the soft support layer 260. The double-sided adhesive layer 285 includes holes that expose portions of the lower surfaces of the conductive gel layers 270, 275, which allow the conductive gel layers 270, 275 to form electric contacts with a user′ skin. The double-sided adhesive layers 280, 285 securely hold the conductive gel layers 270, 275 in the adhesive-conductive assembly 250. The double-sided adhesive layer 280 can be wider than the soft support layer 260 and the double-sided adhesive layer 285 to allow lower adhesive surface of the double-sided adhesive layer 280 to adhere to user's skin, which strengthens the attachment of the compliant wearable patch 200 to user's skin.

In storage, the lower surface of the double-sided adhesive layer 285 is covered by a liner layer (not shown). When the liner layer is peeled off, the lower surface of the double-sided adhesive layer 285 is pressed against a user's skin to be worn by the user at the desired measurement position. The pressure pushes the conductive electrode circuits 230, 235 into the conductive gel layers 270, 275 to form good electric contacts. The conductive electrode circuits 230, 235 each can form conductive contacts with the user's skin respectively through the conductive gel layers 270, 275, which allows the semiconductor chip 222 and other electronic components to measure electrical signals (e.g. ECG signals) in the user's body.

The semiconductor chip 222 and an antenna 225 are configured to wirelessly communicate with external devices, using wireless communication standards such as NFC standard, RFID, Wi-Fi, or Bluetooth. The wireless communication can include data related to the electrical signals picked up by the conductive electrode circuits 230, 235 from the user's body via the conductive gel layers 270, 275, for example, reporting ECG signals to an external device. Examples of external devices include smart phones, computers, mobile payment devices, scanners and readers (e.g. RFID readers), medical devices, security systems, personal identification systems, etc. The antenna circuit can be compatible for NFC communications in a frequency range near 13.56 MHz, as described above, as well as UHF RFID at about 915 MHz, Bluetooth in 2.4 GHz or 5 GHz frequency ranges, and other types of wireless communications.

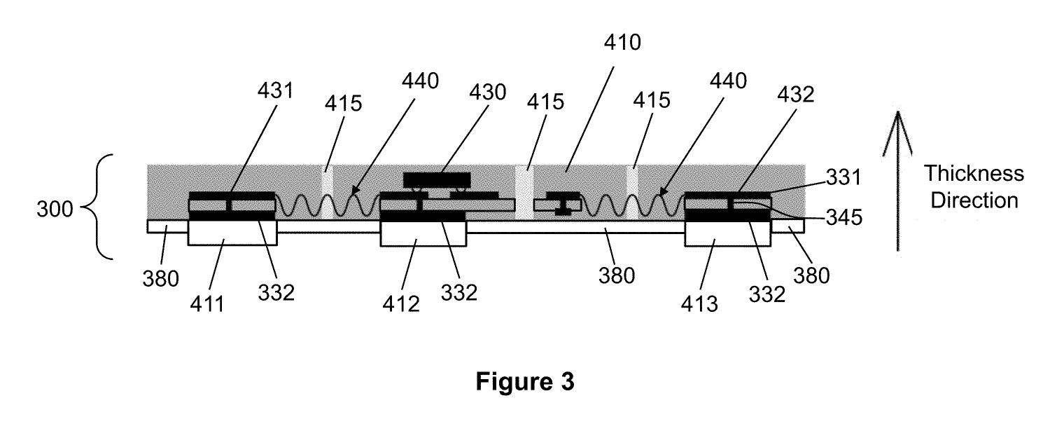

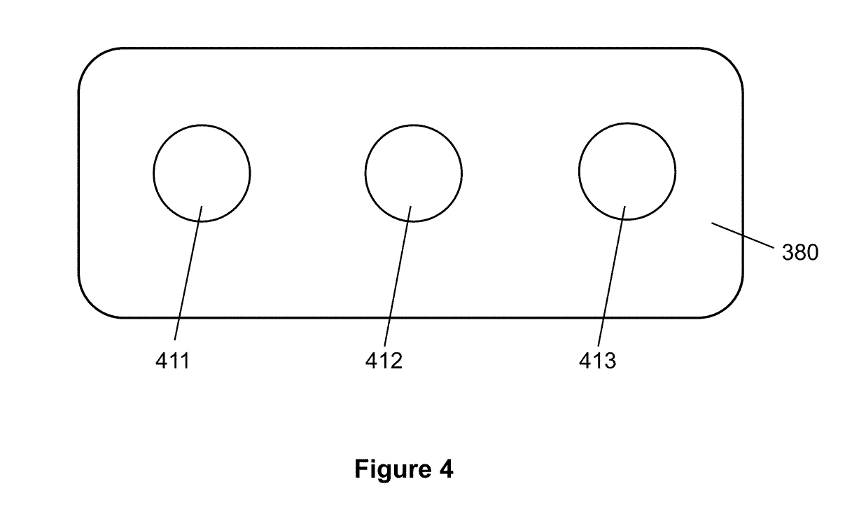

In some embodiments, referring to FIGS. 3 and 4, a compliant wearable patch 300 includes circuit modules 430-432 which are connected by flexible ribbons 540. The circuit modules 430-432 and the flexible ribbons 440 are molded in an elastomer polymer matrix 410. The flexible ribbons 440 include connecting lines configured to transmit electrical signal from the upper electrode layer 331 to the circuit module 430. The circuit module 430, 431 and 432 can include openings and described above. The flexible ribbons 440 can have a curly or serpentine shape or zigzag shape in thickness direction. The openings, the shapes of the flexible ribbons 440 and the elastomer polymer matrix 410 enable the compliant wearable patch to deform easily in the compliant elastomer matrix according to skin deformation with least stress constraints. The compliant matrix material can be formed by a soft material and can be selected from elastomer materials, such as silicone, polyurethane and other elastomer materials. The elastomer polymer matrix 410 can protect chips and circuits from external mechanical damage or electronic discharge to improve reliability in complex environment. The upper electrodes 331 are embedded in the elastomer polymer matrix 410 whereas lower electrodes 332 have their lower surfaces exposed so they can be in contact with the skin. The upper electrodes 331 and the lower electrodes 332 are conductively connected by vias 345. Multiple opening 415 are formed through the elastomer polymer matrix 410 to provide breathability to the skin.

The circuit module 430 is configured to wirelessly communicate with external devices, using wireless communication standards such as NFC standard, RFID, Wi-Fi, or Bluetooth. The wireless communication can include data about the electrical signals picked up by the lower electrode layer 332 from the user's body (e.g. reporting ECG signals to external devices). The circuit module 430 includes a support substrate, and one or multiple semiconductor chips, a circuit, and an antenna circuit formed on or in the support substrate. Examples of external devices include smart phones, computers, mobile payment devices, scanners and readers (e.g. RFID readers), medical devices, security systems, personal identification systems, etc. The antenna circuit can be compatible for NFC communications in a frequency range near 13.56 MHz, as described above, as well as UHF RFID at about 915 MHz, Bluetooth in 2.4 GHz or 5 GHz frequency ranges, and other types of wireless communications.

An adhesive layer 380 is formed on the lower surface of the elastomer polymer matrix 410 but exposing at least portions of the lower surface of each of the lower electrodes 332. The adhesive layer 430 can be laminated to the elastomer polymer matrix 410 for good binding to a user's skin. Layers 411-413 of conductive gel are respectively applied to the lower surfaces of the lower electrodes 332. When a pressure is applied, the layers 411-413 of conductive gel can form good contacts with the lower surface of the lower electrodes 332 and the user's skin, which form a good electric connection between the lower electrodes 332 and the user's skin, which improves the reliability of the compliant wearable patch 300 and measurement accuracy of the electrical signals in human bodies. Examples of suitable materials for the conductive gel include hydrogel and other types of conductive gel.

An elastic layer can be bonded to an upper surface of the elastomer polymer matrix 410 to provide further protection and compliance for the compliant wearable patch 300.

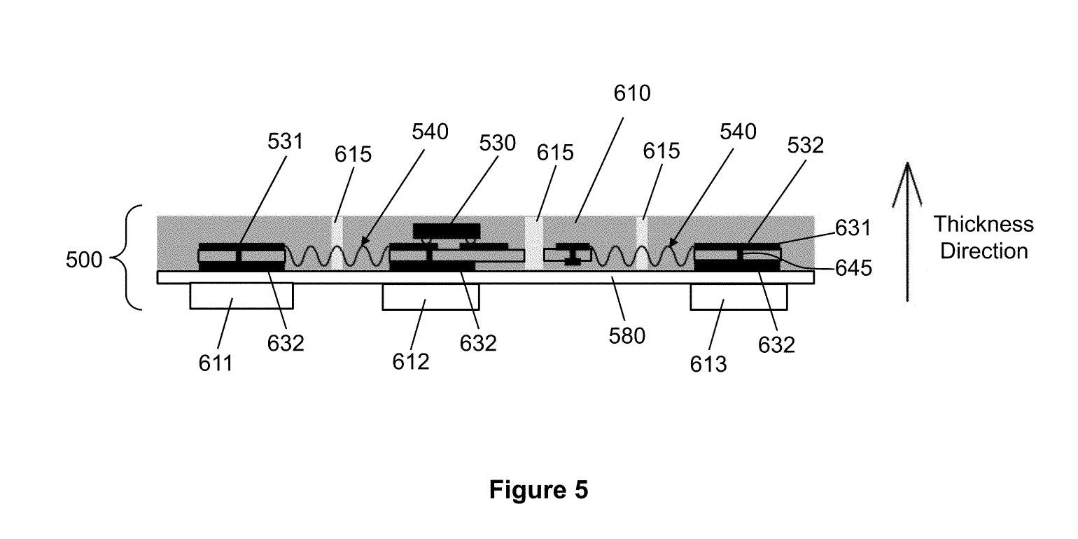

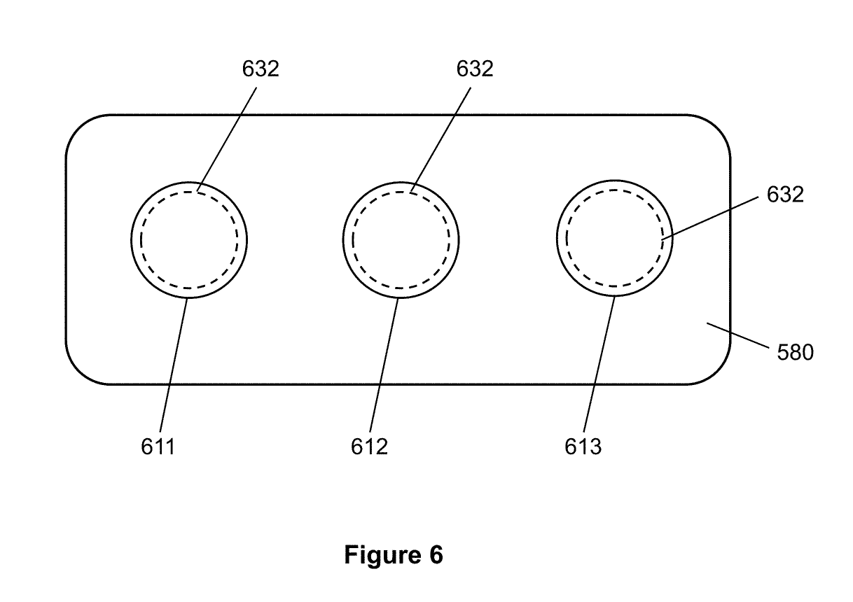

In some embodiments, referring to FIGS. 5 and 6, a compliant wearable patch 500 includes circuit modules 530-532 which are connected by flexible ribbons 540. The circuit modules 530-532 and the flexible ribbons 540 are molded in an elastomer polymer matrix 610. The flexible ribbons 540 include connecting lines configured to transmit the electrical signal from the upper electrode layer 631 to the circuit module 530. The circuit module 530, 531 and 532 can include openings and described above. The flexible ribbons 540 can have a curly or serpentine shape or zigzag shape in thickness direction. The openings, the shapes of the flexible ribbons 540 and the elastomer polymer matrix 610 enable the compliant wearable patch to deform easily in the compliant elastomer matrix according to skin deformation with least stress constraints. The compliant matrix material can be formed by a soft material and can be selected from elastomer materials, such as silicone, polyurethane and other elastomer materials. Multiple opening 615 can be made through the elastomer polymer matrix 610 to give more breathability for skin. The elastomer polymer matrix 610 can also protect semiconductor chips and circuits from external mechanical damage or electronic discharge to improve reliability in complex environment. The upper electrodes 631 are embedded in the elastomer polymer matrix 610 whereas the lower electrodes 632 have their lower surfaces exposed so they can be in contact with user's skin. The upper electrodes 631 and the lower electrodes 632 are conductively connected by vias 645.

A pressure sensitive adhesive layer 580 is uniformly formed on the lower surface of the elastomer polymer matrix 610 including the lower surfaces of the lower electrodes 632. The adhesive layer 580 can be laminated to the elastomer polymer matrix 610 and the lower surfaces of the lower electrodes 632 for good binding to a user's skin. Layers 611-613 of conductive gel are respectively applied to the areas on the lower surface of the adhesive layer 580 that are below the lower surfaces of the lower electrodes 632. Examples of suitable materials for the conductive gel include hydrogel and other types of conductive gel.

Once the compliant wearable patch 500 is pressed against a user's skin, the adhesive layer 580 is firmly attached to the user's skin. The layers 611-613 of conductive gel can form good contacts with the user's skin. The adhesive layer 580 is typically much thinner than the layers 611-613. For example, the layers 611-613 can be around 2 mm or thicker while the adhesive layer 580 is typically 50-500 micron thick. Moreover, the adhesive layer 580 usually has many openings with widths much bigger than the thickness of the adhesive layer 580. Once pressed, the conductive gel in the layers 611-613 can contact the lower surface of the lower electrodes 632 through such openings in the adhesive layer 580. Thus, the layers 611-613 of conductive gel can form a good electric connection between the lower electrodes 632 and the user's skin, which improves the reliability of the compliant wearable patch 500 and measurement accuracy of the electrical signals in human bodies. The adhesive layer 580 can also be pressed onto and peeled off from patch 500. Therefore, in some use cases, the layer 580 together with the layers 611-613 is used as disposable material. A user can replace these layers between uses.

In some embodiments, an elastic layer can be bonded to upper surfaces of the elastomer polymer matrix 610 of the compliant wearable patch 500 to provide further protection and compliance.

The use of conductive gel layers in compliant wearable patches has several advantages. In addition to good electric conduction, the conductive gel in the adhesive-conductive assembly 250 (FIG. 2), the layers 411-413 (FIG. 3), and 611-613 (FIG. 5) is compliant, stretchable, and comfortable to wear on user's skin. The layers 411-413 or 611-613 of conductive gel can easily adapt to the movements of the user's body when the compliant wearable patch 200, 300 or 500 is worn on the user's skin. Moreover, the adhesive-conductive assembly 250 (FIG. 2) can be replaced after a period of usage while the electronic components of the wearable patch 200 can be reused. The adhesive-conductive assembly 250 (FIG. 2) can be separately stored with its outer surfaces protected by liner layers which can be peeled out before bonding to the upper portion of the wearable patch 200 (FIG. 2). The conductive gel can also be separately replaced: after wearing, old layers 411-413 can be peeled off and be replaced by new layers 411-413 of conductive gel (FIG. 3). Old layers 611-613 can be peeled off and be replaced by new layers 611-613 of conductive gel (FIG. 5). The above described replacement feature can reduce cost and increase the lifetimes of the disclosed wearable patches.

While this document contains many specifics, these should not be construed as limitations on the scope of an invention that is claimed or of what may be claimed, but rather as descriptions of features specific to particular embodiments. Certain features that are described in this document in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination.

Only a few examples and implementations are described. Other implementations, variations, modifications and enhancements to the described examples and implementations may be made without deviating from the spirit of the present invention. For example, the usages of the disclosed wearable patches are not limited by the examples given above; they can be applicable to many other fields. The materials suitable for the different layers of the wearable patches are also not limited by the examples provided. The layouts and forms of the elastic layer, the compliant layer, the substrate, the support layer, the adhesive layers, the breathing openings, the semiconductor chip, the antenna, the metal pads, and the connection leads can have other configurations without deviating from the present invention.

What is claimed is:

1. A wearable patch for measuring electrical signals from a user's body, comprising:
a main substrate;
a circuit substrate on the main substrate, the circuit substrate comprising a conductive circuit;
a semiconductor chip in electric connection with the conductive circuit;
a first conductive electrode circuit in electric connection with the conductive circuit, wherein the first conductive electrode circuit includes a portion on a lower surface of the main substrate; and
an adhesive-conductive assembly attached to the lower surface of the main substrate, comprising:
a support layer comprising a first opening;
a first conductive gel layer inserted into the first opening and configured to be in contact with a user's skin;
a first adhesive layer configured to bond to portions of upper surfaces of the first conductive gel layer and a portion of an upper surface of the support layer, wherein the first adhesive layer comprises an upper surface configured to bond to the lower surface of the main substrate, which enables an electric contact to be formed between the first conductive electrode circuit and the first conductive gel layer; and
a second adhesive layer configured to bond to portions of lower surfaces of the first conductive gel layer and a portion of a lower surface of the support layer, wherein the second adhesive layer comprises a lower surface configured to be attached to a user's skin, which allows the first conductive electrode circuit to pick up electric signals from the user's body via the first conductive gel layer.

2. The wearable patch of claim 1, further comprising:
a second conductive electrode circuit in electric connection with the conductive circuit, wherein the second conductive electrode circuit includes a portion on a lower surface of the main substrate,
wherein the support layer includes a second hole opening, wherein the adhesive-conductive assembly further comprises:
a second conductive gel layer inserted into the second opening and configured to be in contact with a user's skin,
wherein the first adhesive layer is further configured to bond to portions of upper surfaces of the second conductive gel layer, wherein the upper surface on the first adhesive layer is configured to bond to the lower surface of the main substrate, which enables an electric contact to be formed between the second conductive electrode circuit and the second conductive gel layer; and
a second adhesive layer further configured to bond to portions of lower surfaces of the second conductive gel layer, wherein the second adhesive layer includes a lower surface configured to be attached to a user's skin, which allows the second conductive electrode circuit to pick up electric signals from the user's body via the second conductive gel layer.

3. The wearable patch of claim 2, wherein the second conductive gel layer protrudes above and below the support layer, wherein the second conductive gel layer includes a lower surface configured to be in contact with a user's skin.

4. The wearable patch of claim 2, wherein the main substrate includes a via through which the second conductive electrode circuit forms a connection between portions on the lower surface and an upper surface of the main substrate.

5. The wearable patch of claim 1, wherein the first conductive gel layer protrudes above and below the support layer, wherein the first conductive gel layer includes a lower surface configured to be in contact with a user's skin.

6. The wearable patch of claim 1, wherein the main substrate includes a via through which the first conductive electrode circuit forms a connection between portions on the lower surface and an upper surface of the main substrate.

7. The wearable patch of claim 1, wherein the first conductive gel layer comprises hydro gel.

8. The wearable patch of claim 1, wherein the upper surface and the lower surface of the second adhesive layer include an adhesive.

9. The wearable patch of claim 1, wherein the upper surface and the lower surface of the first adhesive layer include an adhesive.

10. The wearable patch of claim 9, wherein the first adhesive layer is wider than the support layer to allow the lower surface of the first adhesive layer to attach to a user's skin.

11. The wearable patch of claim 1, wherein the semiconductor chip is configured to wireless communicate with an external device in response to the electric signals picked up by the first conductive electrode circuit from the user's body.

12. The wearable patch of claim 1, further comprising:
a cover formed on the main substrate, portions of the first conductive electrode circuit, and the circuit substrate.
